(19) United States
(12) Patent Application Publication
(10) Pub. No.: US 2021/0009492 A1
(43) Pub. Date: Jan. 14, 2021

(54) DIRECT HYDROXYLATION OF BENZENE TO PHENOL

(71) Applicants: The King Abdulaziz City for Science and Technology, Riyadh (SA); Leibniz Institute for Catalysis at the University of Rostock (LIKAT), Rostock (DE)

(72) Inventors: Mansour Shuleiweeh Alotaibi, Rostock (DE); Venkata Narayana Kalevaru, Rostock (DE); Andreas Martin, Rostock (DE); Mohammed Alkinany, Riyadh (SA); Hamid A Al-Megren, Riyadh (SA)

(73) Assignees: The King Abdulaziz City for Science and Technology, Riyadh (SA); Leibniz Institute for Catalysis at the University of Rostock (LIKAT), Rostock (DE)

(21) Appl. No.: 15/407,489

(22) Filed: Jan. 17, 2017

(51) Int. Cl. C07C 39/04

(52) U.S. Cl. CPC C07C 39/04

(57) ABSTRACT

The invention provides a method for preparing phenols from aromatic compounds, particularly a method of preparing phenol by direct hydroxylation of benzene. The method comprises contacting benzene with nitrous oxide in the presence of a bimetallic zeolite catalyst at an ambient pressure and a reaction temperature ranging from (about) 320° C. to 440° C.

### FIELD OF THE INVENTION

The invention generally relates to a method for preparing phenols from aromatic compounds. More specifically, the invention relates to a method for preparing phenol from benzene by a direct hydroxylation process in the presence of a catalyst.

### BACKGROUND OF THE INVENTION

A number of multi-step as well as the more recent single-step processes for producing phenol from aromatic compounds have been established time and again. More specifically, the use of direct hydroxylation for preparing phenols from aromatic compounds using high reaction temperatures is a known process in the state of art. These high temperature reactions have numerous disadvantages associated with them.

The high reaction temperature hydroxylation methods usually include a very high concentration of N₂O in the reactant feed mixture. The high reaction temperature and high concentration of N₂O in the reactant feed mixture affects the stability of the catalyst systems involved in these reactions which is highly undesirable even though a high yield of phenol is reported. Moreover, the excess of N₂O in the reactant feed affects the important aspect of carbon balance which is more often than not reported of. The excess of N₂O may be easily burnt into a COₓ form further forming various other by-products.

Therefore, there is a need for an improved method for direct hydroxylation of benzene to produce a good yield of phenol without affecting the stability of the reaction.

### BRIEF DESCRIPTION OF THE FIGURES

The accompanying figures, where like reference numerals refer to identical or functionally similar elements throughout the separate views and which together with the detailed description below are incorporated in and form part of the specification, serve to further illustrate various embodiments and to explain various principles and advantages all in accordance with the invention.

FIG. 1 illustrates the direct hydroxylation reaction of benzene to phenol in the presence of bimetallic zeolite catalysts in accordance with an embodiment of the present invention.

### DETAILED DESCRIPTION OF THE INVENTION

Before describing in detail embodiments that are in accordance with the present invention, it should be observed that the embodiments reside primarily in combinations of method steps related to the direct hydroxylation of benzene to phenol. Accordingly, the method steps have been represented where appropriate by conventional symbols in the drawings, showing only those specific details that are pertinent to understanding the embodiments of the invention so as not to obscure the disclosure with details that will be readily apparent to those of ordinary skill in the art having the benefit of the description herein.

In the present disclosure, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element.

The present invention provides an improved process for the production of phenol by direct hydroxylation of benzene using bimetallic zeolite catalysts in a continuous gas phase mode.

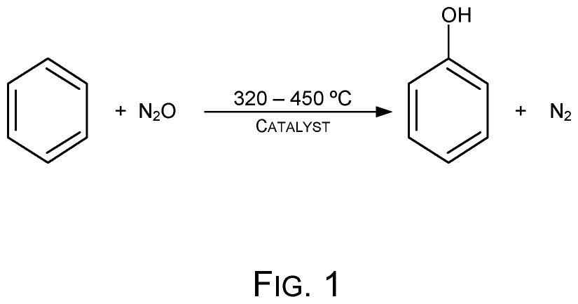

FIG. 1 is illustrative of the direct hydroxylation reaction comprising contacting aromatic compounds with nitrous oxide in presence of a bimetallic zeolite catalyst. In an exemplary embodiment in accordance with the present invention, the direct hydroxylation reaction of benzene to produce phenols, comprises contacting benzene with nitrous oxide in the presence of bimetallic zeolite catalyst at an ambient pressure and a reaction temperature ranging from (about) 320° C. to 440° C. The contacting of aromatic compounds including benzene with nitrous oxide occurs at a contact time of 1 second.

The catalysts used in the present invention are bimetallic zeolite catalysts. The preferred bimetallic zeolite catalysts contain V—Ti catalysts supported on various other different catalysts. The preferred zeolite catalysts include, for example, V—Ti/Ga-ZSM-5, V—Ti/ZSM-5, V—Ti/BEA, V—Ti/USY, etc. In the present invention, the zeolite catalyst forming part of the bimetallic zeolite catalysts are selected from the group of ZSM-5, Ga-ZSM-5, USY and BEA zeolites. The content of V and Ti impregnated in the bimetallic zeolite catalysts comprises 0.25 percentage by weight of Vanadium and 0.25 percentage by percentage of Titanium. The bimetallic zeolite catalysts were calcined in air in a furnace at 500° C.

In an embodiment of the present invention, the method of preparing phenol comprises contacting benzene with the N₂O gas along with N₂ gas for dilution of the reactant feed mixture and for maintaining constant space velocity in a reactor in which the reaction occurs. The overall molar ratio of reactant feed mixture in the reactor is C₆H₆:N₂O:N₂=1:1.5:82.3.

The procedures and advantages of the present invention are illustrated in the following representative examples. However, it is understood that the present invention is not limited to these examples and that any modification and correction can be accomplished within the technical scope of the present invention.

### EXAMPLE 1

The method of preparation of bimetallic zeolite catalysts in accordance with the present invention has been described herein. The supporting zeolite catalysts namely ZSM-5, Ga-ZSM-5, USY and BEA powder were used for preparing various bimetallic zeolite catalysts. The desired quantity of suitable metal precursors as represented in Table 1 was dissolved in distilled water. The solution of metal precursor was impregnated on to the zeolite catalysts (ZSM-5, Ga-ZSM-5, USY and BEA powder) and stirred for 30 min at ambient conditions. The following step included the evaporation of the excess solvent on to a hotplate to dryness. The solid obtained was further dried at 110 ° C. for 12 h in an oven. Finally, the dried solids of bimetallic zeolite catalysts were calcined in air in a furnace at 500° C. at a heating rate of 2 K/min for 6 hrs. The optimum calcination temperature of 500° C. is chosen from 500° C., 600° C. and 700° C. based on maximum conversion of benzene and maximum yield of phenol achieved at 500° C. The composition of the described catalysts, type of metal precursor used and calcination conditions are described below in table 1.

Table 1 is illustrative of V—Ti bimetallic catalysts supported on different zeolite catalysts as given below:

| | | | Calcination |
|---|---|---|---|
| No | Catalyst* | Metal precursor | (° C./h/air) |
| 1 | V—Ti/Ga-ZSM-5 | VCl₃: (NH₄)₂TiO(C₂O₄)₂•H₂O | 500/6 |
| 2 | V—Ti/ZSM-5 | VCl₃: (NH₄)₂TiO(C₂O₄)₂•H₂O | 500/6 |
| 3 | V—Ti/BEA | VCl₃: (NH₄)₂TiO(C₂O₄)₂•H₂O | 500/6 |
| 4 | V—Ti/USY | VCl₃: (NH₄)₂TiO(C₂O₄)₂•H₂O | 500/6 |

### EXAMPLE 2

The experimental setup and catalytic testing procedure of preparing phenol from benzene with N₂O as an oxidizing agent in the presence of a bimetallic zeolite catalyst has been described in accordance with the present invention. The experimental setup comprises a continuously operated gas phase tubular reactor using different variants of a bimetallic zeolite catalyst. The catalytic testing procedure was conducted in a down flow fixed bed stainless steel reactor, wherein 2.0 g (1.0-1.25 mm sieve fraction) of the selected catalyst diluted with corundum was suspended between two quartz wool plugs in the middle of the down flow fixed bed stainless steel reactor. The amount of corundum added to the diluted catalyst is in the ratio of 1:5 by weight. The upper and lower portions of the catalyst stainless steel reactor was also filled with corundum. The N₂O gas along with N₂ gas (for dilution and for maintaining constant space velocity) are supplied to the reactor from compressed gas cylinders at flow rates controlled by mass flow controllers.

The overall molar ratio of reactant feed mixture is C₆H₆:N₂O:N₂=1:1.5:82.3. The reaction was carried out in the temperature range of 410-480° C. The gas hourly space velocity ranges from 2667 to 5333 h⁻¹. Two thermocouples were positioned one at the center of the catalyst bed to indicate reaction temperature and the other thermocouple was attached to furnace through temperature indicator cum controller to monitor the temperature of the reactor. The dosing of benzene to the reactor was done using HPLC pump. Then the reaction temperature was raised to the desired level and the reaction was performed in a continuous manner. The product samples were collected every 30 min and analyzed off-line by GC equipped with both the detectors, i.e. FID (HP-5 column) for liquid products and TCD (Carbon plot column) for gaseous products (e.g. CO_{N}) collected in a gasmouse.

The off-gas volume was measured for each sample and at the same time, three variations of T-profile along the fixed bed was also taken, as illustrated in Table 2 below. The conversion of benzene and the yields of products were calculated in accordance with the standard equations. Phenol is the major product of the reaction. Additionally, some by-products such as catechol, benzoquinone, hydroquinone, COx etc. were also formed along with the major product of phenol.

Table 2 is illustrative of the three variations of T-profile as taken along the bed.

| sample | time | T_{Reaktor}-r | T-Bett. | | | | | | |
|---|---|---|---|---|---|---|---|---|---|
| no. | (min) | (° C.) | (° C.) | T-Profil 1 | | T-Profil 2 | | T-Profil 3 | |
| BOP- | — | — | — | 150° C. | | 410° C. | | 430° C. | |
| 01 | 30 | 150 | 150 | (cm) | (° C.) | (cm) | (° C.) | (cm) | (° C.) |
| 02 | 30 | — | — | — | — | — | — | — | — |
| 03 | 30 | 410 | 410 | 10 | 149.6 | 10 | 409 | 10 | 429 |
| 04 | 30 | — | — | 9 | 150 | 9 | 410 | 9 | 430 |
| 05 | 30 | 430 | 430 | 8 | 149.5 | 8 | 408 | 8 | 428 |
| 06 | 30 | — | — | 7 | 149.4 | 7 | 407 | 7 | 427 |

### EXAMPLE 3

The conversion of benzene and the yield of phenol for the various catalysts have been described in Table 3 below in accordance with the present invention. The mole ratio of the reactant feed mixture namely C₆H₆:N₂O:N₂ is 1:1.5:82.3 at a temperature of 410° C., wherein the benzene flow is at 6 mmol/hour and the reactant mixture is contacted with each other at a contact time of 1.3 seconds. The gas hourly space velocity maintained at 2667 h⁻¹ as per the current example. The weight of the various catalysts as described below was maintained at 2.0 g.

| | Benzene | Phenol |
|---|---|---|
| | conversion | yield |
| Catalysts | (mol %) | (mol %) |
| V—Ti/Ga-ZSM-5 | 33.6 | 27.2 |
| V—Ti/ZSM-5 | 32.1 | 17.7 |
| V—Ti/BEA | 29.8 | 12.7 |
| V—Ti/USY | 4.8 | 0.4 |

### EXAMPLE 4

The effect of reaction temperatures ranging from 410° C. to 480° C. on the catalytic performance of V—Ti/Ga-ZSM-5 has been illustrated in Table 4 below. The mole ratio of the reactant feed mixture namely C₆H₆:N₂O:N₂ is 1:1.5:82.3 wherein the benzene flow is at 6 mmol/hour and the reactant mixture is contacted with each other at a contact time of 1.3 seconds. The gas hourly space velocity was maintained at 2667 h−1 as per the current example. The weight of the various catalysts as described below was maintained at 2.0 g.

Table 4 is illustrative of the effect of reaction temperatures ranging from 410° C. to 480° C. on the catalytic performance of V—Ti/Ga-ZSM-5.

| | Benzene | Phenol |
|---|---|---|
| Temperature | conversion | yield |
| (° C.) | (mol %) | (mol %) |
| 410 | 33.6 | 27.2 |
| 430 | 29.6 | 24.2 |
| 460 | 30.1 | 25.0 |
| 480 | 39.5 | 22.0 |

### EXAMPLE 5

The effect of gas hour space velocity (GHSV) on the catalytic performance of V—Ti/Ga-ZSM-5 catalyst has been illustrated in Table 4 below. The mole ratio of the reactant feed mixture namely C6H6:N2O:N2 is 1:1.5:82.3 wherein the benzene flow is at 6 mmol/hour and the reactant mixture is contacted with each other at a contact time ranging from 0.7 to 1.3 seconds. The gas hourly space velocity was varied between the range of 2667 h⁻¹ to 5333 h⁻¹ as per the current example. The weight of the various catalysts as described below was maintained between 1.0 to 2.0 g.

Table 5 is illustrative of the effect of GHSV on the catalytic performance of V—Ti/Ga-ZSM-5 as shown below.

| | Benzene | Phenol |
|---|---|---|
| GHSV | conversion | yield |
| (h⁻¹) | (mol %) | (mol %) |
| 3555 | 44.0 | 20.5 |
| 5333 | 28.0 | 14.4 |
| 2667 | 33.6 | 27.2 |

Advantageously, the direct hydroxylation reaction of benzene to produce phenol at a specific range of temperature facilitates the maintenance of stability of the various catalysts employed in the reaction as per the invention without compromising on the production yield of phenol. Furthermore, the reactant feed mixture comprises the requisite amount of nitrous oxide thereby maintaining the carbon balance of the reaction and the stability of the reaction along with the stability of the catalysts employed in the reaction.

Those skilled in the art will realize that the above recognized advantages and other advantages described herein are merely exemplary and are not meant to be a complete rendering of all of the advantages of the various embodiments of the present invention.

In the foregoing specification, specific embodiments of the present invention have been described. However, one of ordinary skill in the art appreciates that various modifications and changes can be made without departing from the scope of the present invention as set forth in the claims below. Accordingly, the specification and figures are to be regarded in an illustrative rather than a restrictive sense, and all such modifications are intended to be included within the scope of the present invention. The benefits, advantages, solutions to problems, and any element(s) that may cause any benefit, advantage, or solution to occur or become more pronounced are not to be construed as a critical, required, or essential features or elements of any or all the claims. The present invention is defined solely by the appended claims including any amendments made during the pendency of this application and all equivalents of those claims as issued.

1. A method for direct hydroxylation of aromatic compounds to produce phenols, comprising contacting the aromatic compounds with nitrous oxide in presence of a bimetallic zeolite catalyst including V—Ti bimetallic ions at an ambient pressure and a reaction temperature ranging from 320° C. to 440° C. wherein the bimetallic zeolite catalyst including V—Ti bimetallic ions is selected from a group consisting of V—Ti/ZSM-5, V—Ti/Ga-ZSM-5, V—Ti/BEA or V—Ti/USY.

2. A method for direct hydroxylation of aromatic compounds to produce phenols, comprising contacting benzene with nitrous oxide in presence of a bimetallic zeolite catalyst including V—Ti bimetallic ions, wherein the bimetallic zeolite catalyst including V—Ti bimetallic ions is selected from a group consisting of V—Ti/ZSM-5, V—Ti/Ga-ZSM-5, V—Ti/BEA or V—Ti/USY.

3. The method according to claim 2, wherein the mixture of benzene and nitrous oxide is diluted with nitrogen gas.

4. The method according to claim 3, wherein the molar ratio of C6H6:N2O:N2 is 1:1.5:82.3.

5. (canceled)

6. The method according to claim 1, wherein contacting of aromatic compounds with nitrous oxide occurs at a contact time of 1 second.

7. The method according to claim 1, wherein the conversion of benzene to phenol is in the range of 20 mol percent to 27 mol percent.
